(19) United States
(12) Patent Application Publication
(10) Pub. No.: US 2017/0182318 A1
(43) Pub. Date: Jun. 29, 2017

(54) METHODS OF TREATMENT OF GASTRO-MOTILITY DYSFUNCTION

(71) Applicant: GI Therapies Pty Ltd, Melbourne (AU)

(72) Inventors: David Fisher, Pymble (AU); Bridget Rae Southwell, Gordon (AU)

(21) Appl. No.: 15/072,864

(22) Filed: Mar. 17, 2016

(30) Foreign Application Priority Data
Dec. 23, 2015 (AU) 2015905356

(51) Int. Cl. A61N 1/36; A61N 1/04

(52) U.S. Cl. CPC A61N 1/36007; A61N 1/0484; A61N 1/36014

(57) ABSTRACT

A method of treating a gastro-motility dysfunction in a patient. The method comprises administering transcutaneous, trans-abdominal electrical stimulation to at least two electrodes positioned on an abdominal region of the patient.

### CLAIM OF PRIORITY

This application claims the benefit of priority of Australian Patent Application No. 2015905356, filed on Dec. 23, 2015, the benefit of priority of which is claimed hereby, and which is incorporated by reference herein in its entirety.

### TECHNICAL FIELD

Described embodiments generally relate to methods and systems for the treatment of gastro-motility dysfunction. In particular, described embodiments are directed to methods, devices and systems for transcutaneous, trans-abdominal electrical stimulation to treat one or more gastro-motility dysfunctions.

### BACKGROUND

Gastro-motility dysfunctions such as gastroparesis and functional dyspepsia can severely impair the quality of life of sufferers by causing chronic abdominal symptoms in sufferers. Symptoms of these conditions may include abdominal pain, nausea, vomiting, early satiety, bloating and delayed gastric emptying, and these conditions may lead to malnutrition, anxiety, depression, and early death. These conditions can also cause social and psychological distress for sufferers. Treatment systems exist for treating gastro-motility dysfunctions, often through dietary manipulation and prokinetics, but further treatment options are limited for patients who cannot have these treatments or do not respond to them. Furthermore, the etiology and pathophysiology of gastroparesis and functional dyspepsia are not completely understood.

Treatment systems exist that require providing electrical stimulus via subcutaneously implanted electrodes positioned on the muscle wall of the stomach. However, such treatment systems are undesirably invasive. Further, while such systems may have an immediate effect in assisting with the symptoms of gastro-motility disorders, this effect has not been described as long lasting, or having an effect beyond the immediate time of electrical stimulation.

It is desired to address or ameliorate one or more shortcomings or disadvantages or shortcomings associated with existing gastro-motility dysfunction treatment devices, systems, methods or regimes, or to at least provide a useful alternative thereto.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

Some embodiments relate to a method of treating a gastro-motility dysfunction in a patient, comprising administering transcutaneous, trans-abdominal electrical stimulation to at least two electrodes positioned on an abdominal region of the patient.

In some embodiments, the gastro-motility dysfunction comprises at least one of gastroparesis, functional dyspepsia, small bowel dysmotility, colonic dysmotility, rectal dysmotility and constipation.

In some embodiments, the stimulation is administered simultaneously to four electrodes, at least one of the four electrodes being on a front abdominal region of the patient and at least one of the four electrodes being on aback abdominal region of the patient, to deliver transcutaneous, trans-abdominal electrical stimulation.

For example, in some embodiments, two of the four electrodes are on a front abdominal region of the patient and two of the four electrodes are on a back abdominal region of the patient, to produce multi-angular transcutaneous, trans-abdominal electrical stimulation.

Some embodiments relate to a method of treating a gastro-motility dysfunction in a patient operable in a device configured to generate transcutaneous, trans-abdominal electrical stimulation for delivery to a first set of electrodes on a front of a torso of the patient and a second set of electrodes on a back of the torso, the method comprising:
selecting a first pair of electrodes, wherein the first pair of electrodes comprises one electrode from the first set of electrodes and one electrode from the second set of electrodes;
delivering transcutaneous, trans-abdominal electrical stimulation signals to the selected electrode from the first set of electrodes; and
at the same time as delivering transcutaneous, trans-abdominal electrical stimulation signals to the selected electrode from the first set of electrodes, delivering transcutaneous, trans-abdominal electrical stimulation signals to the selected electrode from the second set of electrodes.

In some embodiments, the method further comprises:
selecting a second pair of electrodes, wherein the second pair of electrodes comprises two electrode from the first set of electrodes; and
simultaneously delivering transcutaneous, trans-abdominal electrical stimulation signals to the selected electrodes from the first set of electrodes and the second set of electrodes, to produce transcutaneous, trans-abdominal electrical stimulation.

In some embodiments, the method further comprises:
selecting a second pair of electrodes, wherein the second pair of electrodes comprises two electrode from the second set of electrodes; and
simultaneously delivering transcutaneous, trans-abdominal electrical stimulation signals to the selected electrodes from the first set of electrodes and the second set of electrodes, to produce transcutaneous, trans-abdominal electrical stimulation.

In some embodiments, the method further comprises:
selecting a second pair of electrodes, wherein the second pair of electrodes comprises one electrode from the first set of electrodes and one electrode from the second set of electrodes; and
simultaneously delivering transcutaneous, trans-abdominal electrical stimulation signals to the selected electrodes from the first set of electrodes and the second set of electrodes, to produce multi-angular transcutaneous, trans-abdominal electrical stimulation.

In some embodiments, the gastro-motility dysfunction comprises at least one of gastroparesis, functional dyspepsia small bowel dysmotility, colonic dysmotility, rectal dysmotility and constipation.

In some embodiments, treatment is administered for at least one treatment period per day over a treatment term of at least one week.

In some embodiments, the treatment term is at least 10 weeks. In some embodiments, the treatment term is between 10 and 20 weeks.

In some embodiments, the treatment period is between about 10 minutes and about 90 minutes. In some embodiments, the treatment period is about 60 minutes.

In some embodiments, at least two electrodes are positioned one to either side of and slightly above an umbilicus on an anterior abdominal wall of a patient, and at least two electrodes are positioned on a thoracic area of T7 to T10 on the patient.

In some embodiments, the electrical stimulation is delivered at a carrier frequency of between about 1 kHz and about 10 kHz, with a modulated frequency of about 20 to about 300 Hz. In some embodiments, the electrical stimulation is delivered at a carrier frequency of about 4 kHz, with a modulated frequency of about 80 to about 150 Hz.

### BRIEF DESCRIPTION OF DRAWINGS

Embodiments are described in further detail below, by way of example and with reference to the accompanying drawings, in which:

FIG. 1 is a schematic diagram illustrating use of electrical stimulation to treat gastro-motility dysfunction in a child, according to some embodiments;

FIG. 2 is a schematic diagram illustrating use of electrical stimulation to treat gastro-motility dysfunction in an adult, according to some embodiments;

FIG. 3A is a schematic illustration showing placement of electrodes in an abdominal region on a person's front side, according to some embodiments;

FIG. 3B is a schematic representation of placement of electrodes in an abdominal region on a backside of the person, according to some embodiments;

FIG. 3C is a schematic plan view illustrating crossing interferential currents between the electrodes, according to some embodiments;

FIG. 4A is a schematic illustration showing placement of multiple frontal electrode pairs, according to some embodiments;

FIG. 4B is a schematic illustration showing placement of multiple posterior electrode pairs, according to some embodiments;

FIG. 5 is a schematic diagram of a belt for assisting electrode placement of single or multiple frontal and posterior electrode pairs, according to some embodiments;

FIG. 6 shows a front view of a stimulation device according to some embodiments;

FIG. 7 shows a stimulation system including the stimulation device of FIG. 6, according to some embodiments;

FIG. 8 shows a flowchart of a method of using the stimulation device of FIG. 6, according to some embodiments;

FIGS. 9A, 9B and 9C show top, perspective and end views of connectors that are configured to plug into the of the device of FIG. 6, according to some embodiments;

FIG. 10 is a diagram illustrating signal path channels of the device of FIG. 6, according to some embodiments; and

FIG. 11 is a diagram illustrating signal paths through a patient, according to some embodiments.

### DETAILED DESCRIPTION

Described embodiments generally relate to methods, systems, devices and treatment regimens for the treatment of gastro-motility dysfunction. In particular, described embodiments are directed to methods, devices and systems for transcutaneous, trans-abdominal electrical stimulation to treat one or more gastro-motility dysfunctions. The stimulation may be trans-abdominal gastrointestinal stimulation in some embodiments.

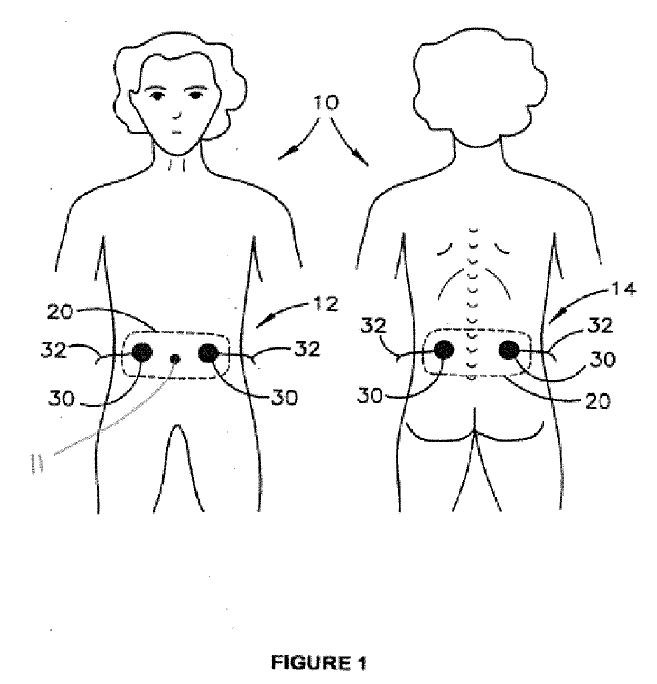

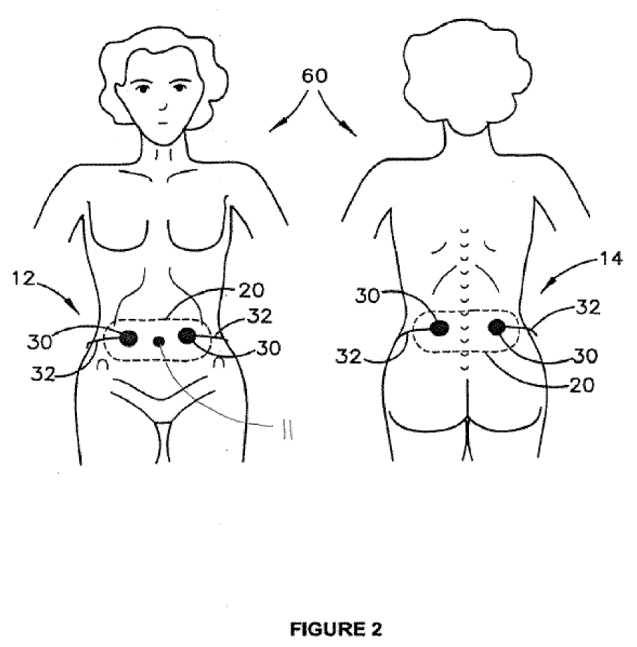

Generally, as illustrated in FIGS. 1, 2, 3A, 3B and 3C in relation to a child 10 or adult 60, the electrical stimulation may be provided to electrodes 30 positioned over a front abdomen region 12 (just above umbilicus 11) and/or on a back (thoracic) abdominal region 14. The electrodes 30 receive electrical stimulation signals via conductors 32 to which they are coupled and convey these to the skin surface of the child 10 or adult 60 to which they are affixed or otherwise conductively positioned against. A suitable conductive gel may be used to increase conductivity of the electrical signals from electrodes 30 into the body via the skin.

In some embodiments, four surface electrodes 30 may be used, two electrodes 30 being positioned one to either side of and slightly above the umbilicus 11 and just below the costal margin on the anterior abdominal wall, and two electrodes 30 being positioned on the thoracic area of T7 to T10. Positioning of the electrodes 30, whether four or more than four electrodes 30 are used, is intended to stimulate the abdominal area, particularly targeting the stomach.

Lateral spacing of the electrode positions from the umbilicus 11 may be in the vicinity of 1, 2, 5 or 8 to 20 cm, for example, thereby providing a lateral separation between the electrodes 30 of about 2, 4, 10 or 15 to 40 cm. Other lateral spacings within such ranges may be employed, as appropriate. The electrodes 30 may be positioned slightly above the umbilicus 11, although some small variation of locations, for example slightly closer to or further from the costal margin, may be employed. The electrodes 30 positioned in the thoracic area may be located substantially directly across the abdomen from the frontal electrodes 30. In some embodiments, the electrodes 30 may be slightly offset from each other vertically or laterally across the pelvis and/or abdomen.

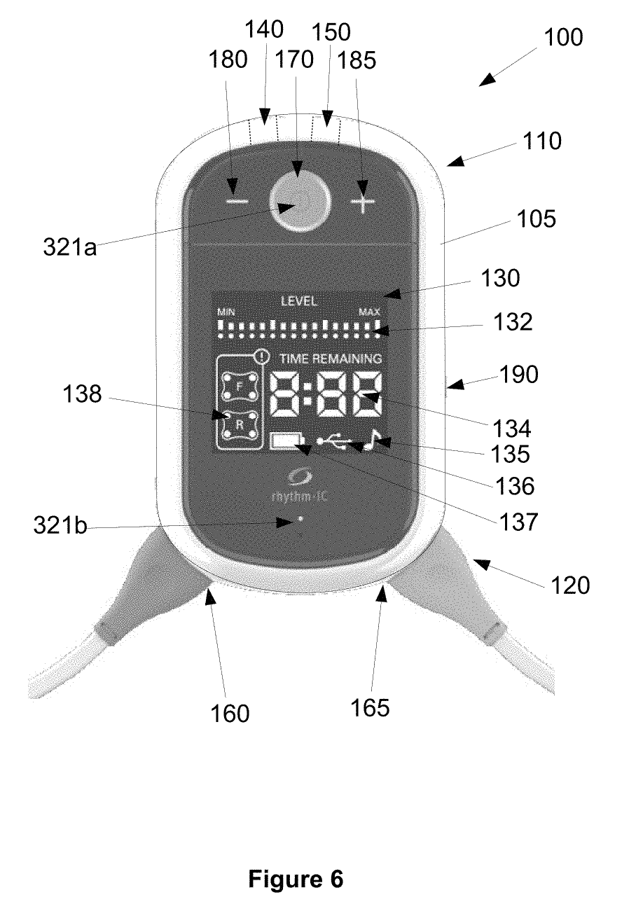

Electrodes 30 may be provided on a carrier 20 that comprises a flexible substrate conveniently positioning the electrodes 30 a fixed distance apart from each other to assist in proper positioning of the electrodes in one or more regions 12, 14. The flexible substrate 20 may comprise adhesive substances on one or more portions thereof in order to facilitate removable application of electrodes 30 to the skin and retention of the electrodes 30 in a specific selected location. Each carrier 20 may comprise 1, 2, 3, 4, 5, 6, 7, 8, 9, 10 or more electrodes 30 in specific spaced relation. Once the electrodes 30 are appropriately positioned, either with or without the aid of a carrier 20, electrode conductor leads 32 are used to couple the conductors 30 to channels of a stimulation device 100 (illustrated in FIG. 6).

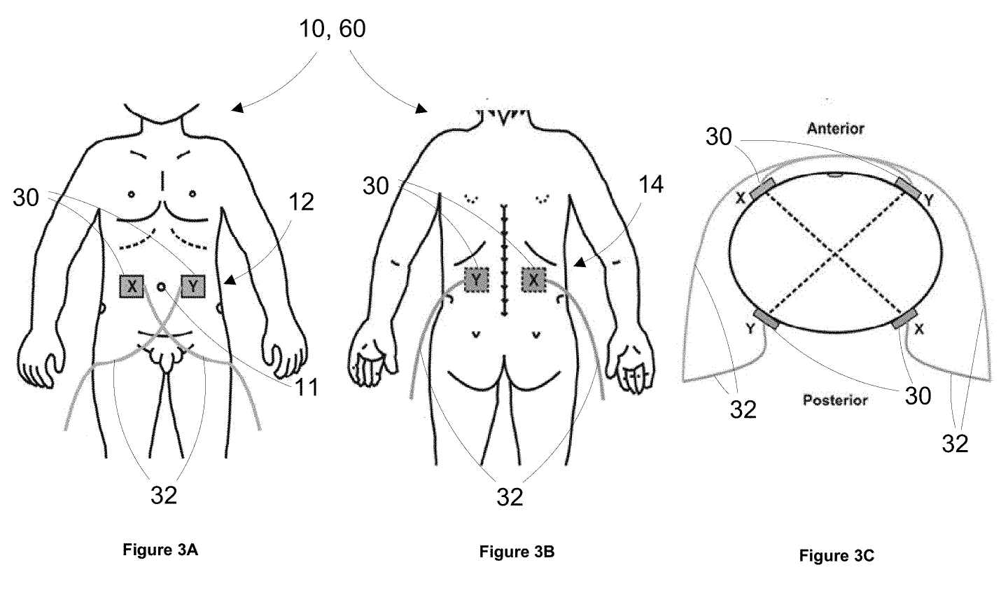

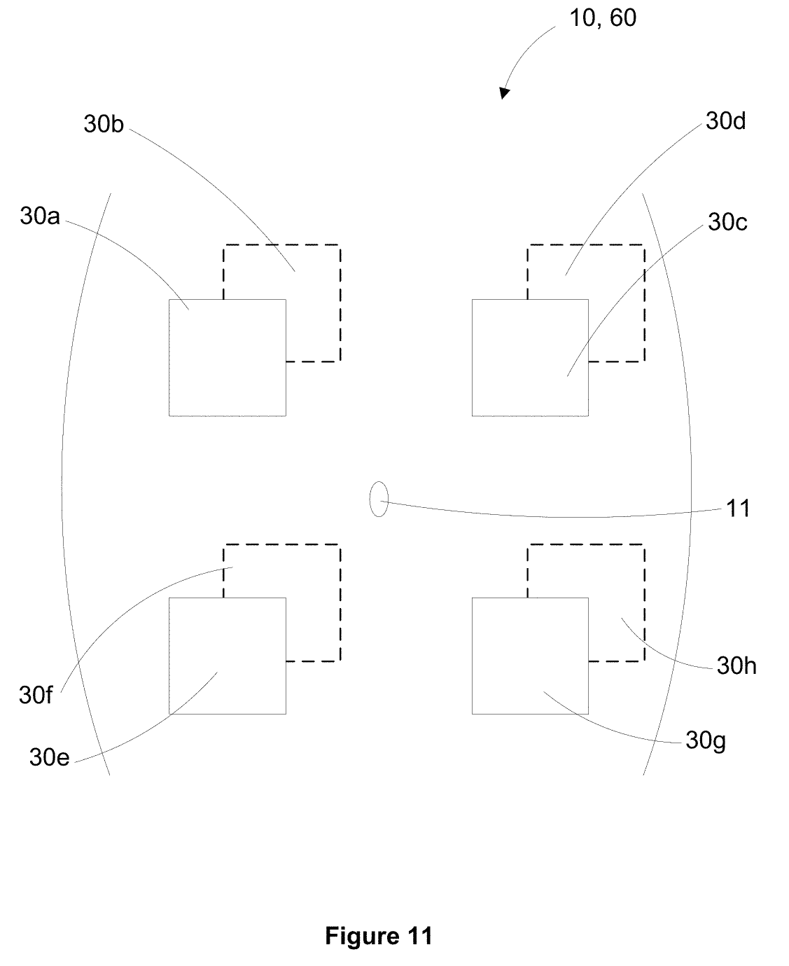

FIG. 3C illustrates schematically how the interferential currents are arranged between front left and back right (X to X) and between front right and back left (Y to Y), such that they provide trans-abdominal stimulation, according to some embodiments. The currents also cross one another, to provide interferential stimulation. In some embodiments, crossing interferential currents may instead be arranged between a front left and back left, and between a front right and back right electrode. A detailed description of further stimulation arrangements can be found below, with reference to FIG. 11. The interferential currents may be provided at two different frequencies, in order to provide interferential current stimulation. Stimulation may be provided at a carrier frequency of between 1 and 10 kHz, with a modulated frequency of about 20 to about 300 Hz. In some embodiments, stimulation may be provided at a carrier frequency of around 4 kHz, with a modulated frequency of about 80 to about 150 Hz. For example, one current may be provided at a frequency of around 4000 Hz, while a second current may be provided to sweep between 4080-4150 Hz.

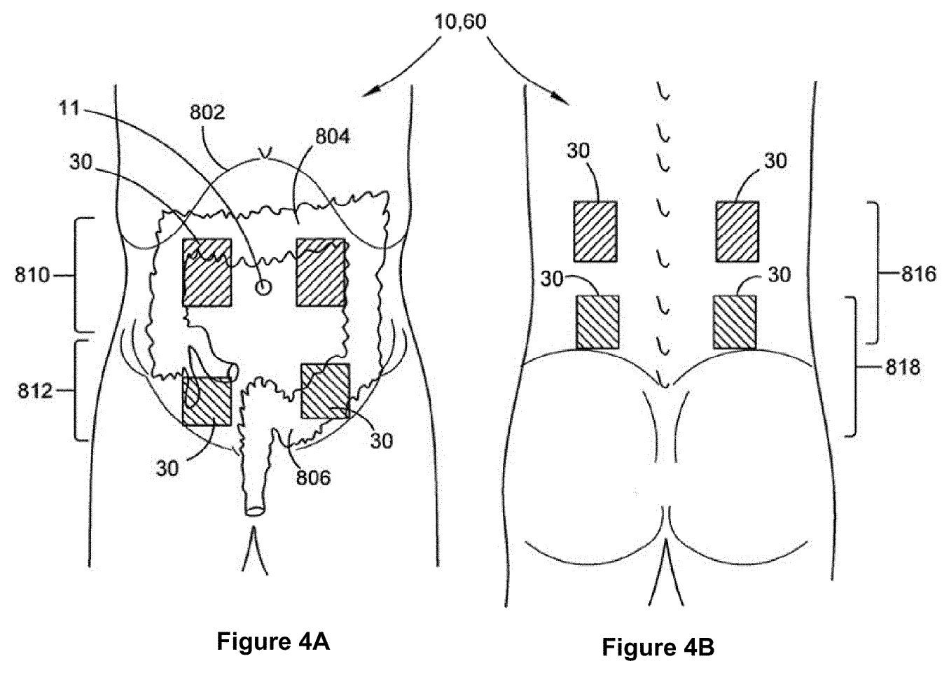

Some embodiments may employ more than two pairs of electrodes, such as the four pairs illustrated in FIGS. 4A and 4B. In such embodiments, the electrode positions illustrated in FIGS. 1, 2, 3A and 3C may be combined with two electrode pairs located below umbilicus 11 in a lower abdominal area. Illustrated electrode pairs 810 and 812 are laterally and vertically spaced in anterior abdominal 810 and lower abdominal 812 areas. Additionally, on the posterior side, two pairs of electrodes 816, 818 are laterally and vertically spaced relative to the spine. The lower pair of posterior electrodes 818 may be positioned above the buttocks on either side of the spine generally opposite the corresponding anterior lower pair 812. The posterior upper pair 816 of electrodes 30 may be positioned generally opposite the corresponding anterior upper pair 810 of electrodes 30 so as to be located in a thoracic area on each lateral side of thoracic vertebrae T7 to T10.

In the embodiments as illustrated in FIGS. 4A and 4B, the electrodes 30 may be operated in two upper pairs and two lower pairs to deliver interferential current stimulation in sequence with one another or simultaneously. Alternatively, the electrodes 30 may be operated in two left pairs and two right pairs to deliver interferential current stimulation in sequence with one another or simultaneously. In some embodiments, the interferential current may be applied between opposed upper and lower electrodes. For example, stimulation current may be applied between one lower posterior electrode 30 and one diagonally opposite upper anterior electrode 30 and optionally also one diagonally opposite lower anterior electrode 30. A detailed description of further stimulation arrangements can be found below, with reference to FIG. 11.

In some embodiments, the upper electrode pairs may be located below the costal margin 802 and generally be positioned to excite or modulate parts of the stomach and ascending 804, transverse and descending 806 colon, while the lower pairs of electrodes 30 may be generally positioned generally in the region T5 to T12. The two upper and two lower pairs of stimulation electrodes 30 are believed to be likely to have a combined positive treatment effect for gastro-motility dysfunction affecting the stomach and abdominal region, such as gastroparesis and functional dyspepsia.

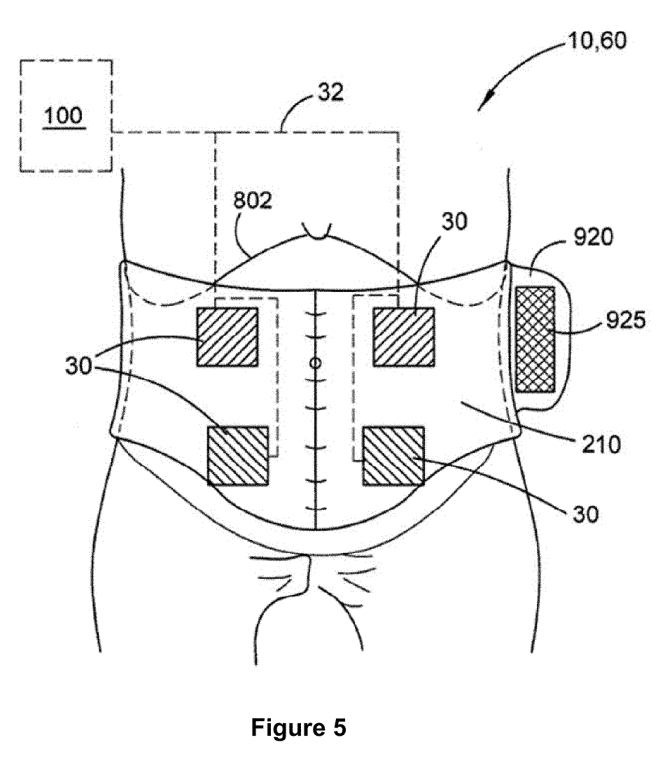

Referring now to FIG. 5, a wearable electrode carrying structure in the exemplary form of a garment 210 is schematically illustrated. Garment 210 may be a belt-like arrangement in some embodiments. An example of a suitable garment 210 is described in International (PCT) patent publication number WO/2015/051405, the entire contents of which is incorporated herein by reference. Garment 210 carries two pairs of anterior electrodes 30 and a further two pairs of posterior electrodes 30 (not shown) to provide stimulation using interferential current delivered from device 100 using conductors 32. Conductors 32 are at least partially supported by the garment 210 and are preferably threaded or passed through at least part of the garment 210 or portions thereof. Garment 210 may comprise carrying means, such as a pocket, pouch, cradle or attachment mechanism, to support and carry the device 100 as the patient moves around.

Although the garment 210 is illustrated as having electrodes 30 positioned to provide stimulation in the manner described in relation to FIGS. 4A and 4B, garment 210 may instead carry a single anterior pair (810/812) and a single posterior pair (816/818) of electrodes for placement in the regions as illustrated in FIGS. 1, 2, 3A and 3C.

In further embodiments, garment 210 may comprise a selectable array of interior electrode connection positions by which the electrode 30 is fixed relative to garment 210 and is in electrical communication with conductors 32 in any of the array of positions. In this way, one or more than one pair of electrodes 30 may be provided anteriorly and one or more than one pair of electrodes 30 can be provided at a selected position or positions posteriorly according to the desired treatment regime prescribed by a medical professional. Once suitable positions of the electrodes 30 on the garment 210 are selected, for example, by the medical professional, the patient 10, 60 can simply place the electrodes 30 in the correct position on their skin for each treatment session by wearing the garment 210 in the same position with respect to the patient's own anatomy.

Garment 210 may be suitably flexible and may be fitted and removed by suitable coupling means, for example such as a side flap 920 having fastening means 925 thereon, such as hook and loop fasteners, buttons or clasps. Garment 210 may be formed of one or more individual or composite layers of flexible (optionally at least partially stretchable) fabric, including for example leather, Lycra, Spandex, cotton, nylon, plastic or other suitable fabric, to provide a wearable garment structure to support the device 100, conductors 32 and electrodes 30. Garment 210 may be generally fluid permeable or impermeable. Preferably, garment 210 is made of one or more machine-washable materials. Garment 210 is preferably sized to be worn with reasonable comfort underneath normal clothing so that a patient wearing the garment 210 can perambulate normally while undergoing the treatment.

In self-administering the treatment prescribed by the medical professional, each patient may be instructed to follow particular instructions for care of the electrodes and their placement in order to maximise effective delivery of the stimulation.

FIG. 6 shows a stimulation device 100 according to some embodiments. Stimulation device 100 comprises a housing 105 with a top end 110, a bottom end 120, and a display portion, which may be a screen such as LCD segment display screen 130 or a touch screen. In some embodiments, screen 130 may be a standard LCD screen, such as a 4.8″ colour LCD screen, for example. Top end 110 and bottom end 120 extend along, and are positioned at opposite ends of, a long axis of the housing 105. Stimulation device 100 may further include other outputs, such as a speaker or piezoelectric buzzer to produce sound, or a motor to provide tactile vibration to the user. Stimulation device 100 may also have inputs such as accelerometers, GPS modules, microphones or cameras. Stimulation device 100 may be a handheld device generally of a size and shape easily held in one hand of the user. The device 100 may further be lightweight and robust, with the exterior being of a smooth construction with few sharp edges or projections. The device 100 may generally be designed for easy portability and handling. A suitable stimulation device 100 is described in detail in International (PCT) patent publication number WO/2015/051406, the entire content of which is incorporated herein by reference.

Screen 130 is configured to be human-readable when stimulation device 100 is oriented such that top end 110 is (from the viewer's perspective) above bottom end 120. In some alternative embodiments, the screen 130 may be configured to be human-readable when stimulation device 100 is oriented such that top end 110 is (from the viewer's perspective) below bottom end 120. In some embodiments, screen 130 may be configured to be human-readable in a different orientation.

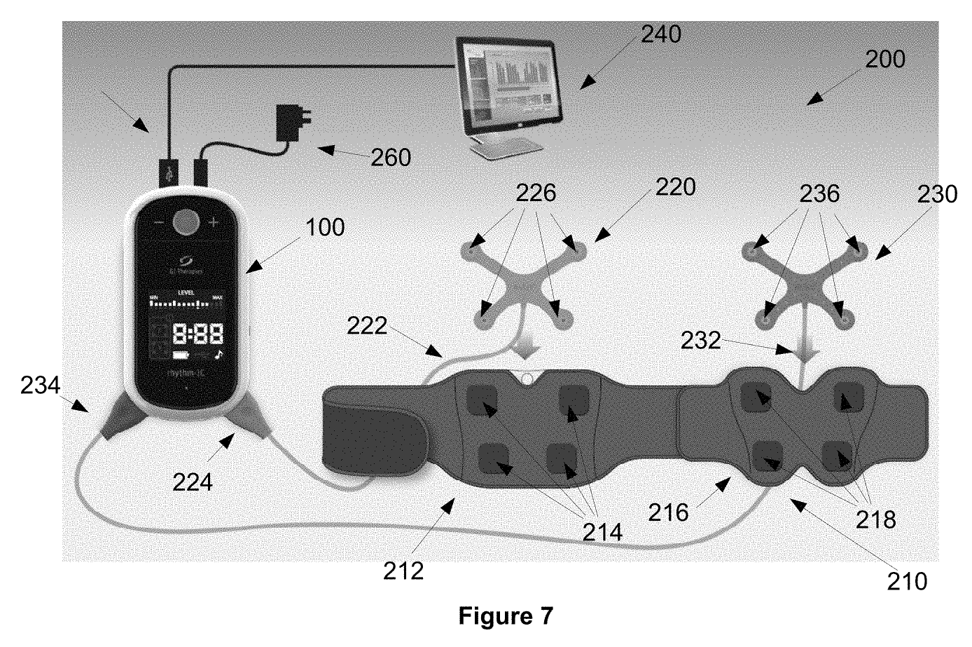

Stimulation device 100 has connector inputs 160 and 165 located at and extending within corner portions of bottom end 120, and may be configured to deliver stimulation signals to electrodes electrically coupled to the connector inputs 160 and 165 (e.g. by connector jacks 224, 234 shown in FIG. 7). In some embodiments, connector inputs 160 and 165 may be positioned at an angle of about 90° to each other. The connector inputs 160 and 165 may respectively comprise sockets to receive respective connector jacks 224, 234 and the housing 105 may define recesses (not shown) at the corner portions of bottom end 120 within which the sockets extend inwardly of the housing 105. Plastic moulded end parts of the jacks can be at least partially received within the recesses. This configuration reduces the risk of the connector jacks coming loose from connector inputs 160 and 165 due to horizontal or vertical strain when the device is worn on the patient's body in the “upside-down” orientation where top end 110 is below bottom end 120.

In some embodiments, connector inputs 160 and 165 may be positioned at another angle to each other, such as an angle of between about 120° and about 60°, or of between about 100° and about 80°. In some embodiments, the angle may be about 90°. In some embodiments, the connector inputs 160 and 165 may be positioned at the corners of the bottom end 120 of device 100.

Stimulation device 100 may further include a clip 195 (not shown) that attaches to a belt or waistband of the user. In some embodiments, the clip may be a loop of wire that extends from bottom end 120 partly down the length of the body of device 100 and back to bottom end 120. The belt may be any conventional belt, or a belt specifically designed to hold stimulation device 100. The waistband may be a waistband of a garment such as a pair of pants or shorts, or a skirt. In some embodiments, another means of attachment to the body of a patient, or to a garment worn on the body of a patient, may be used. This may be a means of attachment to the patient's belt, waistband, or other garment. In some embodiments, the means may include multiple strips of mating Velcro, a series of snaps or buttons. Alternatively, the means of attachment may be a specially-designed belt or holster including a custom pocket designed to receive stimulation device 100 or clip 195. When the device is worn on the body of a patient, the device may be worn such that top end 110 hangs below bottom end 120. When worn in this way, device 100 hangs in an orientation where screen 130 is upside-down (with top end 110 below bottom end 120) and facing away from the body of the user.

To read screen 130, the user can flip device 100 upward about a pivot point, being the flap or portion on garment 210 (FIG. 7) from which device 100 is hanging. Flipping the free end (top end 110) of device 100 in this way allows an orientation of device 100 whereby the screen 130 is facing more towards the body of the user and is right-side up (with top end 110 away from the body and bottom end 120 close to the body) for normal viewing by the user. Thus, the top end 110 can swing freely about its attachment point to garment 210 while the bottom end 120 is held close to the body. When the person wearing garment 210 and the device 100 sits down, there may be a natural tendency for the device 100 to change orientation so that the display screen 130 become inverted enough (from its normal downwardly hanging position) to be readily readable by the person.

Stimulation device 100 may be configured so that when it is being worn in an orientation where top end 110 is held close to the user's body and bottom end 120 is swinging freely below top end 120, the display shown by screen 130 appears upside-down to an observer looking at the user. When stimulation device 100 is flipped up by the user about the pivot at the bottom end 120, the display shown on screen 130 may appear right-side-up to the user as the user glances down at the device 100.

In some embodiments, screen 130 may be configured to change the orientation of the data it displays based on input from an accelerometer, so that any text or images on screen 130 are the correct way up for reading or viewing regardless of the orientation of stimulation device 100.

Stimulation device 100 may include an internal power source, such as a battery pack, allowing it to be portable. Stimulation device 100 may further include a power input 150 to receive the connector of an external power source, which may be a plug pack to be plugged into a mains power source. The external power source may be used as a way of charging the internal power supply. Stimulation device 100 may further include a USB port 140 for the receipt of a USB cable for communication to external computing devices.

Stimulation device 100 may include input means such as buttons, a touch screen, or switches. In some embodiments, stimulation device 100 has a minimal number of buttons, such as only 3, 4 or 5 buttons. This makes the device relatively easy to use and less confusing for the user. In the illustrated embodiment, stimulation device 100 has power button 170, treatment level decrease button 180 and treatment level increase button 185, as well as volume slide switch 190. This allows the user to power the device on and off, to adjust the level of stimulation administered, and to turn the volume of notifications and alarms on and off.

LCD segment display screen 130 provides a visual means of communicating the status of various settings of stimulation device 100 to the user. LCD segment display screen 130 may have between 60 and 90 segments, and in some embodiments may have in the order of 73 segments. Treatment level display bar 132 shows the level of treatment being administered by the device, with the amount of the bar illuminated indicating the relative strength of the stimulation pulses. Time remaining display 134 is made up of several seven segment displays arranged to display a time in hours and minutes. These displays can also be configured to display scrolling characters, which may be used to display messages to the user, such as when a treatment cycle has finished, or a greeting message when the device is powered on, for example. Sound indicator 135 may be illuminated when the sound is turned on by switch 190, and turned off when the sound is off. USB connection indicator 136 may be illuminated when a USB connection is detected through USB port 140, and turned off when there is no USB connection. Battery indicator 137 may be illuminated when the battery power is sensed as being low, indicating that the device battery should be charged by connecting to a power supply through power input 150. In some embodiments, a replaceable battery may be used, and battery indicator 137 may be illuminated when the device battery should be replaced. The battery symbol may be displayed as full during the treatment stage. Pad fault indicator 138 may be illuminated when stimulation device 100 senses that at least one of the front and back electrode connector assemblies 220/230 (see FIG. 7) is disconnected from the device.

Device 100 may further include LEDs as an additional method of visual communication with the user. In the illustrated embodiment, LED 321a may be a green LED used to illuminate power button 170 to indicate that the device is on. LED 321b may be a yellow LED used to indicate that the device is administering treatment. In some embodiments LED 321b may be of a different colour, such as orange.

FIG. 7 shows a stimulation delivery and monitoring system 200 according to some embodiments. System 200 includes stimulation device 100, which is electrically couplable to a back electrode connector assembly 220 and a front electrode connector assembly 230 by back and front electrode connection cables 222 and 232. Back and front electrode connector cables 222 and 232 connect to device 100 through back and front cable connectors 224 and 234, via connector inputs 165 and 160. In some embodiments, the stimulation generation device 100 is arranged to transmit electrical potential to the back and front electrode connector assemblies 220 and 230 via the connector inputs 165 and 160 and the back and front cable connectors 224 and 234.

Back and front electrode connector assemblies 220 and 230 may be attachable to a garment to be worn by a patient being treated. In the illustrated embodiment, the garment may be a belt 210, consisting of a back belt portion 212 and a front belt portion 216, attachable to each other by a temporary fastening means such as Velcro tape, allowing for an adjustment in size to match the body of the wearer. Back and front electrode connector assemblies 220 and 230 each have multiple electrode connectors 226 and 236. In some embodiments, each electrode connector assembly may have two, four or six electrode connectors 226/236. Back and front electrode connectors 226 and 236 are removably couplable to back and front belt portions 212 and 216 by mating snap connector parts (not shown) coinciding with back and front belt electrode placement pads 214 and 218.

Stimulation device 100 may be electrically couplable to an external power source, which may be an AC/DC plug pack 260 to plug into a mains power supply. The external power source may be used to recharge any batteries that are used to operate the device. AC/DC plug pack 260 may be electrically coupled to stimulation device 100 by a cable plugged into power input 150 of the device. Stimulation device 100 may further be electrically couplable to a computing device 240, which may be a desktop computer, laptop computer, tablet, smartphone, or other device. The coupling may be by connection with USB cable 250 between USB port 140 on the stimulation device, and a USB port on computing device 240. This connection may allow for communication between stimulation device 100 and computing device 240 via USB protocol, allowing for the adjustment of treatment settings, and the monitoring of the history of treatment given by stimulation device 100.

In some embodiments, device 100 logs data and records treatment history into an on-chip memory which may later be accessible by computing device 240. Such data may include information about a treatment session, such as the treatment duration, the date the treatment was administered, the time the treatment was administered and the intensity of the stimulation signals delivered during treatment. Communication between stimulation device 100 and computing device 240 may alternatively be achieved through a wireless protocol, such as Bluetooth or Wi-Fi, or through an alternative wired communication protocol. Alternatively, communication between stimulation device 100 and computing device 240 may be effected via an intermediate device, such as a handheld computing device (like a smart phone). Furthermore, the communication may be achieved via a telecommunications network, or via the Internet.

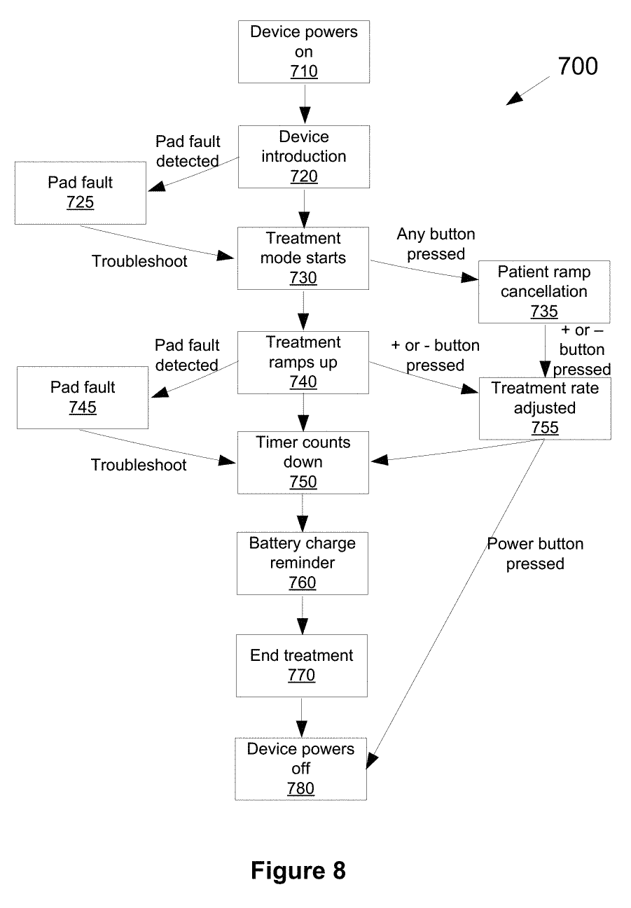

FIG. 8 is a flow chart diagram illustrating method 700 of using stimulation device 100, according to some embodiments. The device is powered up at step 710. In some embodiments, this may be done by holding power button 170 for a set duration of time. In some embodiments, this duration may be in the order of 3 seconds, for example. The device then moves to an introductory display at step 720. This may give the user visual, aural and/or tactile indication that the device has been powered on. For example, the buzzer 322 may be caused to emit a beeping noise, which may continue for a duration of around 1 second in some embodiments. Vibration motor 323 may be activated to cause the device to vibrate briefly. Furthermore or alternatively, the device may be caused to display a message. For example, the word “Hello” may be caused to be displayed and cycled on LCD 130 for a duration of around 4 seconds. LEDs 321 may illuminate. In some embodiments, LED 321a and LED 321b may illuminate, indicating that the device is powered on and that treatment is starting, respectively.

If a pad fault is detected by output monitor component 439, device 100 moves to step 725. Device 100 produces signals to indicate to the user that a fault has occurred. For example, pad fault indicator 138 may be illuminated, buzzer 322 may beep 3 times, and vibration motor 323 may cause a brief vibration. Treatment level display bar 132 and time remaining display 134 are turned off. These indicators prompt the user to troubleshoot, which may be by checking the connections and ensuring cable connectors 234 and 224 are properly plugged into connector inputs 160 and 165. Once the fault is rectified, device 100 progressed to step 730. If no pad fault was detected, device 100 moves directly from step 720 to 730.

At 730, treatment mode commences. This may be a normal treatment mode or a safety treatment or control lock mode, as set up using computing device 240 prior to the use of the device. In treatment mode, device 100 supplies stimulation signals to electrode connectors 226 and 236. Treatment level display bar 132 and time remaining display 134 are illuminating, showing a current treatment level, and the time remaining of the treatment period. The treatment level is the level of the current of the stimulation signals being delivered via electrode connectors 226 and 236. If the user presses any button, treatment ramp is cancelled at 735. Treatment level does not ramp up, but treatment continues at the current level. If the user does not press any buttons, device 100 progresses to step 740, and the treatment level steadily ramps up to a maximum level as set prior to use through computing device 240. In some embodiments, this ramp up may take a period of around 20 seconds, for example. Time remaining display 134 commences counting down the time of treatment remaining. In some embodiments, an initial treatment time of 1 hour may be set, and display 134 may count down each minute until 0 minutes are remaining.

If a pad fault is detected by output monitor component 439, device 100 moves to step 745. Device 100 produces signals to indicate to the user that a fault has occurred. For example, pad fault indicator 138 may be illuminated, buzzer 322 may beep 3 times, and vibration motor 323 may cause a brief vibration. Treatment level display bar 132 and time remaining display 134 are turned off. These indicators prompt the user to troubleshoot, which may be by checking the connections and ensuring cable connectors 234 and 224 are properly plugged into connector inputs 160 and 165. Once the fault is rectified, device 100 may progress to step 750. If no pad fault was detected, device 100 may move directly from step 740 to 750.

If the user presses the treatment level decrease button 180 or the treatment level increase button 185 from either step 735 or 740, device 100 enters step 755 and the treatment level is adjusted up or down depending on the button pressed. In some embodiments, the treatment level is adjusted by 1 level increment each time one of the buttons 180 or 185 are pressed. Some embodiments may have around 40 levels, with a maximum current of around 30 mA into a 1 kΩ load. Each level increment corresponds to about 0.75 or 1 mA for a body having 1 kΩ impedance, so while the change in stimulation intensity is intended to be about 0.75 or 1 mA for each level, it will in practice vary from that amount, depending on the patient receiving the stimulation. In some embodiments, where the device is in a control lock mode (which may be termed a safety mode), the treatment level may increase or decrease a maximum of three levels from the start level (i.e. resulting in a maximum intensity variation from the start level of around 2 mA to 3 mA). Each time a button 180 or 185 is pressed, the user may receive feedback that the button press was registered by device 100. For example, treatment level display bar 132 may display the new treatment level, and time remaining display 134 may be used to display a numeric treatment level for a period, which may be in the order of 2 seconds in some embodiments. Where device 100 is in safety mode or control lock mode, device 100 may give the user a visual, aural or tactile indication of when the maximum treatment level has been reached. For example, buzzer 322 may emit a short beep, and vibration motor 323 may cause device 100 to vibrate briefly. After a period of no buttons being pressed, which may be a period of 5 seconds, for example, device 100 moves from step 755 to 750. Alternatively, if the user holds power button 170 for a period, which may be a period of 3 seconds in some embodiments, device 100 may move to step 780 and power off.

From step 750, the treatment continues at the level reached during ramp up or set by the user, and the treatment time continues to count down. At step 760, when the treatment time is nearing to 0, such as when it reaches 1 minute, battery indicator 137 may flash to remind the user to charge the device. When the countdown reaches 0, at step 770, treatment ends. This may be indicated to the user in a number of ways. For example, buzzer 322 may emit 1 long beep, corresponding to vibration motor 323 vibrating for one long period. In some other embodiments, buzzer 322 may emit 2 short beeps and 1 long beep, corresponding to vibration motor 323 vibrating for 2 short periods followed by one longer period. Time remaining display 134 may be used to display a message to the user, which may be the word “End” in some embodiments. The device may stay switched on for a period of time, which may be 3 minutes in some embodiments, before automatically switching off. Alternatively, the user may be able to power off the device by holding power button 170 for a period of time, such as a period of 3 seconds, for example.

In some embodiments, the user of the application may be able to adjust aspects of the stimulation signals such as the duty period, duty cycle, sweep delta minimum, sweep delta maximum, sweep period, switching mode, and output switch over frequency values. This enables a wide variety of stimulation signals and electrode switching patterns to be set up through the application running on computing device 240 as required.

Referring again to FIG. 7, in some embodiments of system 200, electrode connectors 226 and 236 may be arranged in upper and lower banks of electrode connectors. For example, there may be two electrode connectors in the upper bank and two in the lower bank on each electrode connector assembly 220 and 230, such that when the device is worn, the upper electrode connectors sit against the patient's upper abdomen and the lower bank electrode connectors sit on the patient's lower abdomen. In this case, device 100 may be configured to enable the stimulation signals on the lower bank of electrodes for 15 minutes, then on the upper bank for 15 minutes, followed by 15 minutes on the lower bank and finishing with 15 minutes on the higher bank. These configurations can be changed to any other combination of stimulation patterns through firmware. For example, in some embodiments, device 100 may activate the upper bank of electrodes first for a first set period, and subsequently activate the lower bank for a second set period, which may be the same or different from the first set period. The stimulation periods can be adjusted to be longer or shorter in duration as required. In some embodiments, electrodes may alternatively be configured in left and right banks, such that device 100 may be configured to activate only the right bank of electrodes, only the left bank, or to switch between right and left banks.

In some alternative embodiments of system 200, there may only be a single bank of electrodes, and the device may be worn so that the electrodes are placed on either the upper or the lower abdomen of the patient, for example as described with reference to FIGS. 1 to 3C.

A detailed description of further stimulation arrangements can be found below, with reference to FIG. 11.

Stimulation may be administered for at least one treatment session per day over consecutive days of a treatment period of at least one week. The treatment session may be performed multiple times per day or just once and may be performed for a time between about 10 and about 90 minutes for each session. In some embodiments, the treatment session may be between about 20 minutes and about 60 minutes, preferably closer to 60 minutes, such as 25, 30, 35, 40, 45, 50, 55, 65 or 70 minutes or other times in between. Other time periods include about 11, 12, 13, 14, 15, 16, 17, 18, 19, 21, 22, 23, 25, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 61, 62, 63, 64, 65, 66, 67, 68, 69, 70, 71, 72, 73, 74, 75, 76, 77, 78, 79, 80, 81, 82, 83, 84, 85, 86, 87, 88 and 89 minutes, for example.

In some embodiments, daily treatments are anticipated of greater than 12 sessions in a 4 20 week period (i.e. three sessions per week). Reference to “greater than 12 sessions” includes from about 12 to about 100 sessions such as about 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 sessions, for example, or even more. For ease of administration, the treatment device is such that it can be used at home without the supervision of a trained healthcare professional during the treatment regimen.

The treatment term of at least one week may be, for example, between about 2 weeks and about 5 months. This includes periods of about 3, 5 4, 5, 6, 7, 8, 9, 10, 11, 12, 13, 14, 15, 16, 17, 18, 19 and 20 weeks, for example. In some embodiments, the treatment term may be between about 2 months and about 4 months.

The treatment term may be repeated over an extended term of from about 4 months to 2 years, in order to have the treatment suitably program, teach or train the various muscles or nerves responsible for proper function of the affected organs or tissues. Such longer-term periods include about 4, 5, 6, 7, 8, 9, 10, 11, 12, 13, 14, 15, 16, 17, 18, 19, 20, 21, 22, 23 and 24 months, for example. Thus, the treatment period may be repeated multiple times over the longer term, with the degree of repetition depending on physiological response to one or more initial treatment terms.

The treatment may have an effect beyond the immediate time of electrical stimulation which may last 1 day, to 1 week, to 1 month to one year beyond the last time of electrical stimulation.

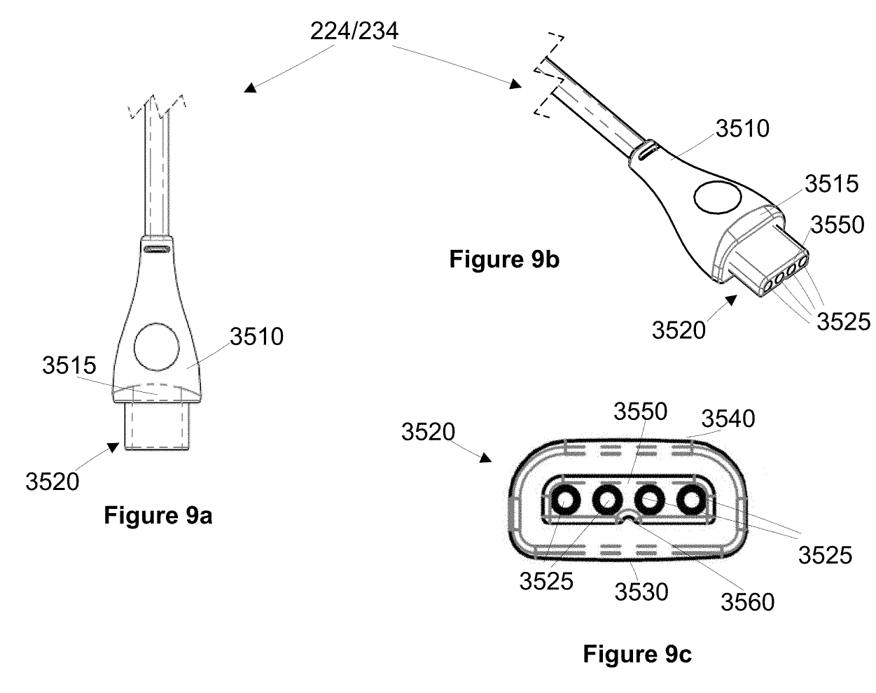

FIG. 9A shows a top view of cable connector 224 or 234 of the system of FIG. 7. Although system 200 has back cable connector 224 and front cable connector 234, only one cable connector is illustrated for simplicity. Back and front cable connectors 224 and 234 may each have a shaped gripping portion 3510 and connector plug portion 3520. Shaped gripping portion 3510 may be moulded of plastic or otherwise formed to a size and shape for easy manual handling. The shape may be designed to be fitted to the exterior of the external housing of device 100, such that when connectors 224 and 234 are inserted into connector inputs 160 and 165, gripping portion 3510 limits the degree of movement of connector 224 or 234 with respect to device 100 about the insertion axis of connector 224 or 234. Device 100 may have a recessed area around each connector input 160 and 165 to receive and couple with a part of the exterior of gripping portion 3510 that is adjacent the connector pin 3520. In some embodiments, back and front cable connectors 224 and 234 may be identical in shape, allowing either connector to be plugged into either connector input 160 or 165. In some embodiments, back and front cable connectors 224 and 234 may be mirror images of each other, or otherwise different in shape, with each recess on the housing of device 100 coupling only to one of connectors 224 or 234, to prevent each connector 224 or 234 from being plugged into the wrong connector input 160 or 165.

Connector plug portion 3520 may be an electrically conductive plug portion to be received in and to electrically couple with connector input 160 or 165.

Connector plug portion 3520 may extend out of the gripping portion 3510 and contain a series of hollow pin port plugs 3525. Each pin port plug may form part of a separate electrically isolated signal channel. In some embodiments, device 100 may have 2, 3, 4, 5, 6, or another number of electrically isolated signal channels, each corresponding to a pin port 3525.

In some embodiments, the connector plug portion 3520 may be a four pin port plug and each pin port 3525 may be associated with and arranged to electrically couple to a respective pin of the stimulation generation device 100 to thereby allow transcutaneous electrical stimulation to be delivered to the electrode connector assembly 220, 230.

Pin ports 3525 may be arranged linearly along a latitudinal axis of the connector plug 3520. Alternatively, pin ports 3525 may be aligned along a longitudinal axis of the connector plug 3520. In other embodiments, pin ports 3525 may be arranged in any suitable configuration, for example, in a square or circular formation.

In some embodiments, the connector plug portion 3520 may comprise a front face 3550 and the pin ports 3525 may extend through the front face 3550. Connector plug portion 3520 may include a notch 3560 for aligning connector plug portion 3520 with a corresponding projection in stimulation generation device 100. In this way, the connector plug portion 3520 may be received in stimulation device 100 only when it is orientated correctly.

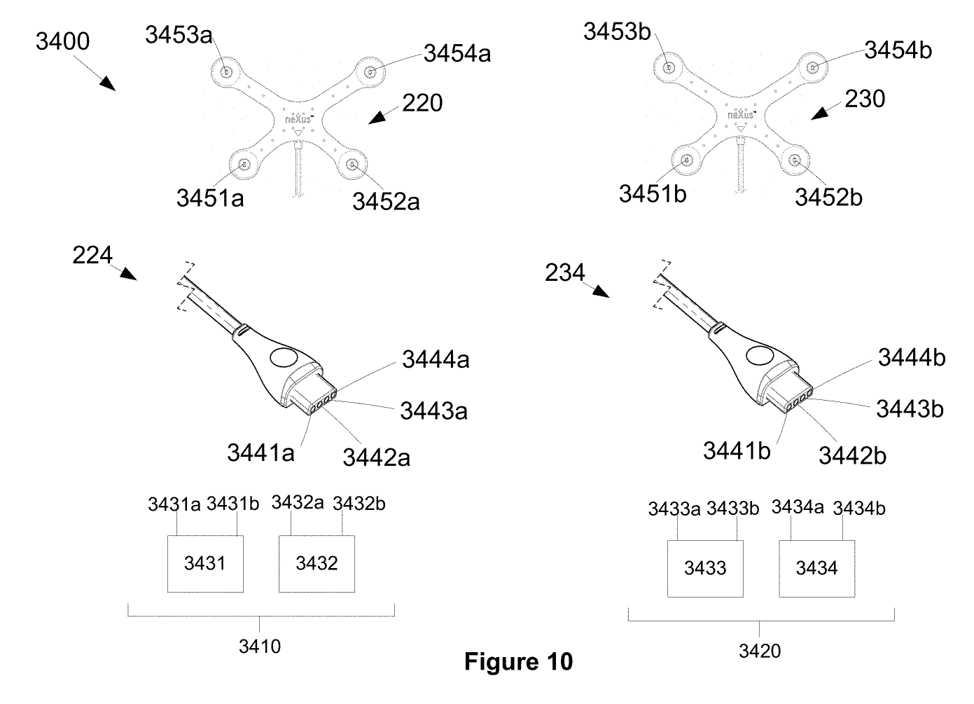

FIG. 10 is a diagram of the signal path channels 3400 of the device of FIG. 6. As described with reference to FIG. 7 above, output stage 380 of device 100 drives output transformers that supply current to electrode connectors 226 and 236. In some embodiments, the transformers may include four transformers 3431, 3432, 3433 and 3434, one for each back/front electrode pair. In some embodiments, these may be divided into lower transformer bank 3410, having transformers 3431 and 3432, and upper transformer bank 3420 having transformers 3433 and 3434. In some embodiments, the transformers may alternatively be divided into a left transformer bank and a right transformer back, or divided in some other manner into sets of transformer banks. Each transformer output may be split into an “a” and “b” line. The output signals from each transformer may be passed through connectors 224 and 234 and onto electrode connector assemblies 220 and 230. In some embodiments, all “a” lines are passed to front connector 224, while all “b” lines are passed to back connector 234.

In some embodiments, connector 224 may have conductive pin sections 3441a, 3442a, 3443a and 3444a, and connector 234 may have conductive pin sections 3441b, 3442b, 3443b and 3444b. Conductive pin section 3441a, 3442a, 3443a and 3444a may be electrically coupled to lines 3431a, 3432a, 3433a and 3434a, respectively. Conductive pin section 3441b, 3442b, 3443b and 3444b may be electrically coupled to lines 3431b, 3432b, 3433b and 3434b, respectively.

Connector 224 is electrically coupled with front electrode connector assembly 220, having electrode connector portions 3451a, 3452a, 3453a and 3454a. In some embodiments, conductive pin section 3441a, 3442a, 3443a and 3444a may be electrically coupled to electrode connector portions 3451a, 3452a, 3453a and 3454a, respectively. Connector 234 is electrically coupled with back electrode connector assembly 230, having electrode connector portions 3451b, 3452b, 3453b and 3454b. In some embodiments, conductive pin section 3441b, 3442b, 3443b and 3444b may be electrically coupled to electrode connector portions 3451b, 3452b, 3453b and 3454b, respectively.

For each electrode connector assembly 220 or 230, signals coming from lower transformer bank 3410 appear on electrodes placed in the lower section of electrode connector assembly 220 or 230, while signals coming from upper transformer bank 3420 appear on electrodes placed in the upper section of electrode connector assembly 220 or 230. Therefore, in some embodiments, when worn on the human body front and back electrode connector assemblies 220 and 230 provide stimulation to the upper section of the patient's abdomen or the lower section of the patient's abdomen at any given time, but cannot stimulate both simultaneously. Device 100 may be configured to provide stimulation to both upper and lower electrode sets by switching the active transformer bank between activating lower bank 3410 and upper bank 3420. This switching may be done periodically (e.g. every 10 to 20 minutes), or after a programmed interval (e.g. after about 10, 15, 20, 25, 30, 35, 40, 45 or 50 minutes).

In some embodiments, microcontroller 355 of device 100 may be configured to control the generation and delivery of interferential current stimulation signals to different pairs of electrodes that include any electrode to which back electrode connector assembly 220 is connected in combination with any electrode to which front electrode connector assembly 230 is connected. This is further described below with reference to FIG. 8. For example, microcontroller 355 may be configured to control the generation and delivery of stimulation signals between an electrode electrically connected to the top left of back electrode connector assembly 220 and an electrode electrically connected to the bottom right of front electrode connector assembly 230. In another example, device 100 may be configured to deliver stimulation signals to an electrode electrically connected to the top left of back electrode connector assembly 220 and an electrode electrically connected to the bottom left of front electrode connector assembly 230 simultaneously. Alternatively, any other pair of electrodes, wherein one electrode is selected from each of the electrodes electrically connected to back electrode connector assembly 220 and front electrode connector assembly 230, may be controlled by microcontroller 355 to deliver stimulation signals based on stored configuration parameters in device 100. This may be done by providing stimulation signals to a pair of conductive pin sections, wherein one conductive pin section is selected from connector 224 and one conductive pin section is selected from connector 234. In use, two such selected electrode pairs may receive interferential current stimulation signals from the stimulation device 100 and apply these signals transcutaneously across parts of the abdominal region.

A successful treatment of a patient by administration of transcutaneous, trans-abdominal electrical stimulation treatment delivered using the device 100 of some of the described embodiments is one that may achieve at least one or more of the following results: (a) nausea has decreased; (b) vomiting has decreased; (c) the feeling of post prandial fullness has diminished; (d) the feeling of early satiety has diminished; (e) abdominal pain has decreased; (f) the rate of gastric emptying has improved; (g) the rate of transit through the small intestine and/or colon is improved; (h) frequency of defecation has improved; (i) soiling has improved; (j) stool consistency has improved; (k) presence of stool in the rectum has reduced; (l) rectal wall strength has improved; and (m) diameter of rectum has reduced.

FIG. 11 shows a diagram of conductive electrodes 30 placed on a patient 10/60. In some embodiments, electrodes 30a, 30c, 30e and 30g may be placed on the front of the torso of patient 10/60, while electrodes 30b, 30d, 30f and 30h may be placed on the back of a torso of patient 10/60. In some embodiments, electrodes 30a, 30c, 30e and 30g may correspond to electrode connector portions 3451a, 3452a, 3453a and 3454a, respectively, of a front electrode connector 220, and electrodes 30b, 30d, 30f and 30h may correspond to electrode connector portions 3451a, 3452a, 3453a and 3454a, respectively, of a back electrode connector 230.

According to some embodiments, gastro-motility dysfunction treatment may be administered by applying transcutaneous, trans-abdominal electrical stimulation to two or more electrodes 30 positioned on an abdominal region of patient 10/60. The two or more electrodes include at least one electrode positioned on the front of the torso of patient 10/60, and at least one electrode positioned on the back of the torso of patient 10/60. For example, in some embodiments, treatment may include providing stimulation to at least one of electrodes 30a, 30c, 30e and 30g, together with at least one of electrodes 30b, 30d, 30f and 30h.

According to some embodiments, gastro-motility dysfunction treatment may be administered by applying transcutaneous, trans-abdominal electrical stimulation to four or more electrodes 30 positioned on an abdominal region of patient 10/60. The four or more electrodes include at least two electrodes positioned on the front of the torso of patient 10/60, and at least two electrodes positioned on the back of the torso of patient 10/60. For example, in some embodiments, treatment may include providing stimulation to at least two of electrodes 30a, 30c, 30e and 30g, together with at least two of electrodes 30b, 30d, 30f and 30h. This may produce multi-angular transcutaneous, trans-abdominal electrical stimulation. For example, stimulation may be supplied simultaneously to the following electrode pairs: 30a to 30d and 30b to 30c; 30e to 30h and 30f to 30g; 30b to 30g and 30d to 30e; 30c to 30h and 30d to 30g; 30b to 30e and 30 a to 30f; and 30a to 30h and 30c to 30f.

According to some embodiments, gastro-motility dysfunction treatment may be administered by applying transcutaneous, trans-abdominal electrical stimulation to multiple different sets of electrodes 30 for different periods of time. In some embodiments, stimulation may be applied to two or more sets of electrodes 30 in an alternating sequence. For example, stimulation may be applied to electrodes 30a to 30d and 30b to 30c for a particular duration, and then applied to electrodes 30e to 30h and 30f to 30g for a second duration. This sequence may be repeated for a predetermined period of cycles to make up a treatment session. In some embodiments, each set of electrodes 30 may be activated at least once during a treatment cycle.

Stimulation device 100 may be programmable to deliver stimulation based on any of the stimulation patterns described above, or any other stimulation pattern.

Studies involving some described embodiments are described by the following non-limiting example.

### Example 1

The Use of a System for Delivering Transcutaneous, Trans-Abdominal Electrical Stimulation in the Treatment of Individuals with a Gastro-Motility Dysfunction Condition.

Patient Group:

6 individuals suffering from gastroparesis that has failed to respond significantly to medical treatments such as dietary modifications, oral and rectal laxatives. Given the diversity of human body shapes, individuals participating were of a variety of body shapes and sizes associated with weight, age, ethnicity and gender.

Stimulation Regime:

Stimulation was applied using 4 electrodes, positioned just above the umbilicus to thoracic level T7 to T10 to target the stomach. 2 electrodes were positioned on the front of the torso, and 2 were positioned on the back of the torso, as illustrated in FIGS. 3A and 3C. A stimulation frequency of 4000 Hz was applied to one channel, and 4080-4150 Hz was applied to a second channel of crossing electrodes, as illustrated in FIG. 3B. Stimulation was applied for 1 hour per day for 11 to 17 weeks.

Results:

Nausea, vomiting and bloating improved or ceased in all patients. Those requiring total parenteral nutrition or enteral nutrition prior to treatment were able to establish normal feeding regimes. Normal bowel movements were obtained by 4 of the 6 patients.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

What is claimed is:

1. A method of treating a gastro-motility dysfunction in a patient, comprising administering transcutaneous, trans-abdominal electrical stimulation to at least two electrodes positioned on an abdominal region of the patient.

2. The method of claim 1, wherein the gastro-motility dysfunction comprises at least one of gastroparesis, functional dyspepsia, small bowel dysmotility, colonic dysmotility, rectal dysmotility and constipation.

3. The method of claim 1, wherein the stimulation is administered simultaneously to four electrodes, at least one of the four electrodes being on a front abdominal region of the patient and at least one of the four electrodes being on a back abdominal region of the patient, to deliver transcutaneous, trans-abdominal electrical stimulation.

4. The method of claim 3, wherein two of the four electrodes are on a front abdominal region of the patient and two of the four electrodes are on a back abdominal region of the patient, to produce multi-angular transcutaneous, trans-abdominal electrical stimulation.

5. A method of treating a gastro-motility dysfunction in a patient, the method operable in a device configured to generate transcutaneous, trans-abdominal electrical stimulation for delivery to a first set of electrodes on a front of a torso of the patient and a second set of electrodes on a back of the torso, the method comprising:
selecting a first pair of electrodes, wherein the first pair of electrodes comprises one electrode from the first set of electrodes and one electrode from the second set of electrodes;
delivering transcutaneous, trans-abdominal electrical stimulation signals to the selected electrode from the first set of electrodes; and
at the same time as delivering transcutaneous, trans-abdominal electrical stimulation signals to the selected electrode from the first set of electrodes, delivering transcutaneous, trans-abdominal electrical stimulation signals to the selected electrode from the second set of electrodes.

6. The method of claim 5, further comprising:
selecting a second pair of electrodes, wherein the second pair of electrodes comprises two electrode from the first set of electrodes; and
simultaneously delivering transcutaneous, trans-abdominal electrical stimulation signals to the selected electrodes from the first set of electrodes and the second set of electrodes, to produce transcutaneous, trans-abdominal electrical stimulation.

7. The method of claim 5, further comprising:
selecting a second pair of electrodes, wherein the second pair of electrodes comprises two electrode from the second set of electrodes; and
simultaneously delivering transcutaneous, trans-abdominal electrical stimulation signals to the selected electrodes from the first set of electrodes and the second set of electrodes, to produce transcutaneous, trans-abdominal electrical stimulation.

8. The method of claim 5, further comprising:
selecting a second pair of electrodes, wherein the second pair of electrodes comprises one electrode from the first set of electrodes and one electrode from the second set of electrodes; and
simultaneously delivering transcutaneous, trans-abdominal electrical stimulation signals to the selected electrodes from the first set of electrodes and the second set of electrodes, to produce multi-angular transcutaneous, trans-abdominal electrical stimulation.

9. The method of claim 5, wherein the gastro-motility dysfunction comprises at least one of gastroparesis, functional dyspepsia, small bowel dysmotility, colonic dysmotility, rectal dysmotility and constipation.

10. The method of claim 1, wherein treatment is administered for at least one treatment period per day over a treatment term of at least one week.

11. The method of claim 10, wherein the treatment term is at least 10 weeks.

12. The method of claim 11, wherein the treatment term is between 10 and 20 weeks.

13. The method of claim 10, wherein the treatment period is between about 10 minutes and about 90 minutes.

14. The method of claim 13, wherein the treatment period is about 60 minutes.

15. The method of claim 1, wherein at least two electrodes are positioned one to either side of and slightly above an umbilicus on an anterior abdominal wall of a patient, and at least two electrodes are positioned on a thoracic area of T7 to T10 on the patient.

16. The method of claim 1, wherein the electrical stimulation is delivered at a carrier frequency of between about 1 kHz and about 10 kHz, with a modulated frequency of about 20 to about 300 Hz.

17. The method of claim 16, wherein the electrical stimulation is delivered at a carrier frequency of about 4 kHz, with a modulated frequency of about 80 to about 150 Hz.

18. The method of claim 5, wherein treatment is administered for at least one treatment period per day over a treatment term of at least one week.

19. The method of claim 5, wherein at least two electrodes are positioned one to either side of and slightly above an umbilicus on an anterior abdominal wall of a patient, and at least two electrodes are positioned on a thoracic area of T7 to T10 on the patient.

20. The method of claim 5, wherein the electrical stimulation is delivered at a carrier frequency of between about 1 kHz and about 10 kHz, with a modulated frequency of about 20 to about 300 Hz.
